(19) United States
(12) Patent Application Publication
(10) Pub. No.: US 2020/0277649 A1
(43) Pub. Date: Sep. 3, 2020

(54) BROAD SPECTRUM DETECTION OF DNA DAMAGE BY REPAIR ASSISTED DAMAGE DETECTION (RADD)

(71) Applicants: University of South Alabama, Mobile, AL (US); RAMOT AT TEL AVIV UNIVERSITY, LTD., Tel-Aviv (IL)

(72) Inventors: Natalie Rose Gassmann, Mobile, AL (US); Nathaniel Holton, Mobile, AL (US); Yuval Ebenstein, Yavne (IL)

(73) Assignee: Ramot at Tel Aviv University, Ltd, Tel-Aviv (IL)

(21) Appl. No.: 16/645,101

(22) PCT Filed: Sep. 6, 2018
(86) PCT No.: PCT/US2018/049689

§ 371 Date: Mar. 6, 2020

Related U.S. Application Data
(60) Provisional application No. 62/556,073, filed on Sep. 8, 2017.

(51) Int. Cl. C12Q 1/68; C12Q 1/34

(52) U.S. Cl. CPC C12Q 1/68; C12Q 1/34

(57) ABSTRACT

The present invention relates to methods and compositions for detection of DNA damage, e.g., in a sample, cell or organism, that are amenable to high throughput, efficient and/or economical implementation, also capable of assessing a broad spectrum of DNA lesions.

### CROSS-REFERENCE TO RELATED APPLICATIONS

This application claims the benefit of priority under 35 U.S.C. § 119 to U.S. Provisional Patent Application Ser. No. 62/556,073, entitled “BROAD SPECTRUM DETECTION OF DNA DAMAGE BY REPAIR ASSISTED DAMAGE DETECTION (RADD)”, which was filed Sep. 8, 2017. The entire contents of this patent application are hereby incorporated by reference herein.

### FIELD OF THE INVENTION

The invention relates generally to methods and compositions for identification of DNA damage, e.g., in a sample, cell or organism.

### BACKGROUND OF THE INVENTION

Environmental exposures, reactive by-products of cellular metabolism, and spontaneous deamination events result in a spectrum of DNA adducts that, if un-repaired, threaten genomic integrity by inducing mutations, increasing instability, and contributing to the initiation and progression of cancer. Assessment of DNA adducts in cells and tissues is critical for genotoxic and carcinogenic evaluation of chemical exposure and may provide insight into the etiology of cancer. Numerous methods to characterize the formation of DNA adducts and their retention for risk assessment have been developed. However, there are still significant drawbacks to the implementation and wide-spread use of these methods, because they often require a substantial amount of biological sample, highly specialized expertise and equipment, and depending on technique, may be limited to the detection and quantification of only a handful of DNA adducts at a time. Thus, a need exists for high throughput and/or easy to implement assays that can assess a broad spectrum of DNA lesions.

### BRIEF SUMMARY OF THE INVENTION

The current disclosure relates to discovery of an assay for detecting nuclear DNA lesions in cells, which can broadly detect damage across species and tissue types. The assay, termed “Repair Assisted Damage Detection” or “RADD” herein, is highly adaptable and, in certain embodiments, includes: (1) a DNA damage processing mix containing DNA repair enzymes that recognize, remove, and modify DNA lesion sites to contain the appropriate DNA end chemistry for gap filling, and (2) a gap filling mix containing a tagged nucleotide to be incorporated for monitoring the processed DNA damage site. In certain embodiments, the DNA repair enzymes that are utilized by RADD can be adjusted depending upon the DNA lesions being investigated, and the tagged nucleotide can also be altered depending upon the read-out that is desired. The RADD method described herein therefore provides users with a high content screening assay capable of monitoring a broad spectrum of DNA damage, with no DNA isolation required.

In one aspect, the instant disclosure provides a composition for detecting and processing DNA adducts contained in damaged DNA, the composition including a plurality of two or more of the following enzymes: uracil DNA glycosylase (UDG), formamidopyrimidine [Fapy]-DNA glycosylase (FPG), T4 pyrimidine dimer glycosylase (T4PDG), endonuclease IV (Endo IV), and endonuclease VIII (ENDOVIII), provided that one of the plurality of enzymes is Endo IV.

In one embodiment, the composition includes at least three of the enzymes. In a related embodiment, the composition includes at least four of the enzymes. Optionally, the composition includes all five of the enzymes.

Another aspect of the disclosure provides a kit for conducting repair assisted Damage detection (RADD), e.g., in a cell, upon an array of fixed cells, etc., where the kit includes a composition of the disclosure, and instructions for its use.

In one embodiment, each enzyme is contained in a separate container.

In another embodiment, each enzyme is contained in the same container.

In one embodiment, the RADD kit further includes a gap filling mix, optionally present in a separate container from, e.g., an above-recited composition of the disclosure, where the gap fill mix includes a Klenow DNA polymerase and deoxy Uracil Triphosphate (dUTP) which may be directly or indirectly conjugated to a label.

Another aspect of the disclosure provides a method of detecting and quantifying damage to nuclear DNA that includes: (a) fixing and permeabilizing cells; (b) treating the fixed and permeabilized cells with a composition for detecting and processing DNA adducts which includes a plurality of two or more enzymes selected from UDG, FPG, T4PDG, ENDOIV, and ENDOVIII, provided that one of the plurality of enzymes is ENDOIV, where the treating recognizes and processes DNA adducts contained in the nuclear DNA, thereby allowing for incorporation of a labeled dUTP into the site at which the adduct had occurred (e.g., into a gapped DNA site, at the site of single strand break, at a double strand break site, etc.); (c) treating the cells of (b) with a gap filling mix that includes a Klenow DNA polymerase and dUTP, where the dUTP is labeled, or a label is affixed thereto following incorporation of the dUTP into the damaged nuclear DNA; and (d) detecting and quantifying the DNA damage as a function of intensity of the label by any recognized method for detecting label, optionally using a fluorescent read-out, e.g., microscopy, plate readers, flow cytometry, etc.

In one embodiment, the cells are human cancer cells, i.e., A375P, U2OS, HCT116 cells, or other eukaryotic cells, i.e., CHO-K1 cells.

In another embodiment, prior to the fixation, the cells are incubated in a hypotonic solution. In a related embodiment, the hypotonic solution includes a cytoskeleton buffer (CSK). Optionally, after fixation the permeabilizing includes incubating the thus-incubated cells in a mild permeabilization buffer.

In one embodiment, step (b) of the above method includes incubating the fixed and permeabilized cells with UDG, FPG, T4PDG, ENDOIV, and ENDOVIII.

In another embodiment, the dUTP is linked to biotin, and optionally where the detecting and quantifying in step (d) includes incubating the cells of (c) with an anti-biotin antibody, and then quantifying the DNA damage as a function of the intensity of the label that is indirectly conjugated to the anti-biotin antibody. In a related embodiment, the label is a fluorescent dye, and the quantifying comprises measuring the intensity of the fluorescent signal emitted by the dye.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

By “agent” is meant any small compound (e.g., small molecule), antibody, nucleic acid molecule, or polypeptide, or fragments thereof.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art.

As used herein, the term “mild permeabilization buffer” refers to a solution capable of acting as a buffer containing a low percentage of surfactant (e.g., 0.01-1.0% Triton X-100, optionally 0.1-0.25% Triton X-100) that is applied to cells for 5-10 mins at room temperature (i.e., approximately 23-25° C.).

The term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

As used herein, the term “diagnosing” refers to classifying pathology or a symptom, determining a severity of the pathology (e.g., grade or stage), monitoring pathology progression, forecasting an outcome of pathology, and/or determining prospects of recovery.

The term “in vitro” as used herein refers to events that occur in an artificial environment, e.g., in a test tube or reaction vessel, in cell culture, etc., rather than within a multi-cellular organism.

As used herein “in vivo” refers to events that occur within a multi-cellular organism, such as a human and a non-human animal. In the context of cell-based systems, the term may be used to refer to events that occur within a living cell (as opposed to, for example, in vitro systems).

The terms “isolated,” “purified,” or “biologically pure” refer to material that is free to varying degrees from components which normally accompany it as found in its native state.

“Isolate” denotes a degree of separation from original source or surroundings. “Purify” denotes a degree of separation that is higher than isolation.

By “marker” is meant any protein or polynucleotide having an alteration in expression level or activity that is associated with a disease or disorder.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, the term “tumor” means a mass of transformed cells that are characterized by neoplastic uncontrolled cell multiplication and at least in part, by containing angiogenic vasculature. The abnormal neoplastic cell growth is rapid and continues even after the stimuli that initiated the new growth has ceased. The term “tumor” is used broadly to include the tumor parenchymal cells as well as the supporting stroma, including the angiogenic blood vessels that infiltrate the tumor parenchymal cell mass. Although a tumor generally is a malignant tumor, i.e., a cancer having the ability to metastasize (i.e., a metastatic tumor), a tumor also can be nonmalignant (i.e., non-metastatic tumor). Tumors are hallmarks of cancer, a neoplastic disease the natural course of which is fatal. Cancer cells exhibit the properties of invasion and metastasis and are highly anaplastic.

Unless specifically stated or obvious from context, as used herein, the term “or” is understood to be inclusive. Unless specifically stated or obvious from context, as used herein, the terms “a”, “an”, and “the” are understood to be singular or plural.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIG. 1 schematically illustrates a RADD workflow diagram, beginning with a damage event or endogenous basal DNA damage followed by fixation and permeabilization. Cells were then incubated with DNA damage processing mix (UDG, FPG, T4PDG, Endo IV, Endo VIII), followed by co-incubation with the DNA gap filling mix (Klenow DNA polymerase, biotin-dUTP). Cells were visualized after incubation with anti-biotin-FITC conjugated antibody and fluorescence confocal microscopy (scale bar=50 μm).

FIG. 2 depicts that whole cell RADD signal was increased by treatment with CSK buffer. A375P cells that were fixed and permeabilized using three different methods followed by RADD assay readout. The panel of confocal images shown were representative of undamaged A375P melanoma cells (scale bar=50 μm).

FIGS. 3A to 3C show that RADD could detect UVC- and KBrO₃-induced DNA damage. FIG. 3A shows a survival curve for A375P cells that were treated with increasing doses of KBrO₃ (1 h), while FIG. 3B shows a survival curve for A375P cells that were treated with increasing doses of UVC. Such cells were grown until control, untreated cells reached approximately 95% confluency. Graphs are representative of the percent survival relative to the untreated control. FIG. 3C shows bar graphs and imaging results obtained when A375P cells were treated with sub-lethal doses of UVC or KBrO₃, and the RADD assay was then performed to detect DNA lesions normalized to the undamaged control. Image panels were representative of cells quantified to create the bar graphs. All graphs were constructed from three independent experiments, with error bars representing the standard error of the mean (SEM) (scale bar=25 μm).

FIGS. 4A and 4B depict RADD-mediated detection of KBrO₃-induced lesions over time. The RADD assay was performed upon A375P cells treated with 10 mM KbrO₃ for 1 h, which were then allowed to recover for the indicated time points. The bar graphs of FIG. 4A were constructed from more than 350 cells per time point and error bars represent the SEM. The corresponding images of FIG. 4B were representative of each time point that was analyzed by RADD.

FIGS. 5A and 5B show that RADD was able to detect BrdU incorporation and DNA damage induced by laser micro-irradiation. In FIG. 5A, CHO cells were sensitized with BrdU for 24 hours, and BrdU incorporation was then detected by RADD. The bar graph was constructed by measuring the nuclear intensity per area of four independent BrdU sensitization events, with representative images adjacent to the bar graph. The SEM was calculated from >1500 cells and was +9.46 and 5.56 mean intensity/area, respectively, for the bar graphs of FIG. 5A. In FIG. 5B, laser micro-irradiation was performed upon CHO cells that were sensitized with 10 μM BrdU for 24 h. Cells were fixed 5 minutes post irradiation using a 405 nm laser stimulation (2.4 mW) and were then probed with monoclonal antibodies or RADD. Scale bar represents 10 μm.

FIG. 6 depicts a multiplexed RADD workflow diagram.

FIGS. 7A and 7B show lesion induction by KBrO₃ and UVC in A375P cells. In FIG. 7A, A375P cells were treated with 20 mM KBrO₃ (1 h) and allowed to recover for the indicated time points. Detection of DNA adducts in the genomic DNA was performed by immunoslot blot with antibodies against 8oxodG. In FIG. 7B, A375P cells were treated with 20 J/m2 UVC and allowed to recover for the indicated time points. Detection of DNA adducts in the genomic DNA was performed by immunoslot blot with antibodies against CPD.

FIG. 8 shows detection of endogenous biotin. The background level of biotin staining was assessed by performing the RADD assay of the instant disclosure in the absence and presence of Klenow DNA polymerase. When no Klenow was present, anti-biotin signal accumulated in the cytoplasm with very poor nuclear fluorescent signal. When Klenow was present with the full complement of RADD enzymes and biotinylated dUTP, the nuclear signal was significantly increased over the cytoplasmic signal. The nucleus was counterstained with DAPI.

### DETAILED DESCRIPTION OF THE INVENTION

The present invention is directed, at least in part, to discovery and development of a DNA damage detection assay, herein termed the Repair Assisted Damage Detection (RADD) assay, which harnesses the action of specific DNA repair enzymes to recognize and excise DNA adducts throughout the genome. Once the DNA adduct has been removed, the adduct position is tagged by insertion of a biotinylated deoxyuridine triphosphate (dUTP). This detection method has herein been designed and demonstrated to be effective in applying the method to cells (e.g., fixed cells) to measure DNA damage in situ.

### DNA Damage Detection

Nucleic acids are continuously subjected to modification by endogenous and exogenous sources. The formation and retention of these nucleic acid modifications or adducts can threaten the fidelity of the genome by altering the nucleic acid structure, changing base pairing and promoting the likelihood of insertions, deletions and translocations. Detection and removal of DNA adducts is essential for maintaining genomic fidelity, and a tailored and lesion specific DNA damage response (DDR) has evolved for the enzymatic recognition of DNA adducts and the coordination of repair by a suite of DNA repair pathways. Mutations in genes involved in DDR are linked to aging and genetic diseases, as well as cancer predisposition, and these mutations can also alter treatment outcomes (1-4). Therefore, assessment of DNA adduct formation and persistence in cells aids in the determination of the genotoxic or carcinogenic potential of chemical or environmental exposures and may identify subpopulations vulnerable to exposure effects. This potential has led to the development of assays that monitor and measure the formation and retention of DNA adducts within a genome, in order to assess the functional DNA repair capacity (reviewed in (5-7)).

Liquid chromatography and mass spectrometry have been used extensively to identify and quantify DNA adducts. These methods have allowed precise quantitation of adduct levels from cell models to patients and have significantly advanced our understanding of the structure and lifetime of DNA adducts. However, these techniques require expert users, expensive equipment, often employ isotopic labeling for precise quantitation, and require microgram quantities of isolated DNA (6, 8, 9). While there are distinct advantages to utilizing these techniques to measure specific adducts, there are issues with DNA isolation procedures introducing further DNA damage and in the standardization of measurements (10).

More accessible forms of DNA damage and adduct detection are antibody based strategies, comet assays, and enzymatic detection by terminal deoxynucleotidyl transferase (TdT). Antibody strategies can be applied to isolated DNA, in cells, or in fixed tissues. While antibodies exist for strand break signals (γH2AX or 53BP-1) and some DNA lesions (thymine dimer, cyclobutane pyrimidine dimers (CPD), etc.), these techniques are limited by the small number of highly specific antibodies that have been validated and may be difficult to multiplex due to incompatibilities in fixation or staining procedures. Comet assay or Single Cell Gel Electrophoresis allows more specific strand break detection in cells, eliminating the requirements for specific antibodies, and with modifications can detect alkali labile sites, oxidative base damage, and DNA cross-linking (11, 12). However, comet assay has been difficult to standardize and reproduce from lab to lab, though comet chip technologies and automated image processes are improving these shortcomings (13-15).

Terminal deoxynucleotidyl transferase dUTP nick end labeling (TUNEL) and in situ (DNA) end labeling (ISEL; 16-18)) have also been employed extensively over the past 20 years to detect DNA strand breaks during apoptosis and in some cases DNA damage across a variety of biological samples (19-21). However, just like the other methods, there are drawbacks to using TUNEL or ISEL because they are highly specific for 3′-OH ends. Several TUNEL modifications have emerged extending its ability to detect other types DNA ends (i.e., 3′-PO₄) or improve DNA damage detection by incorporating FPG to excise oxidative DNA adducts (22).

While all of these techniques are used extensively in the literature to assess DNA damage and adduct formation, each has significant limitations for broad spectrum detection of DNA damage.

### Repair Assisted Damage Detection (RADD)

The RADD assay described herein provides a platform for the characterization of DNA damage repair (DDR) within and across different cell lines by scoring the DNA lesion load. It has been identified herein that RADD is a robust assay for the measurement of DDR and can be used to investigate specific DNA repair mechanisms, address risk assessment for both environmental toxicology and cancer etiology, and evaluate DNA targeted cancer therapies.

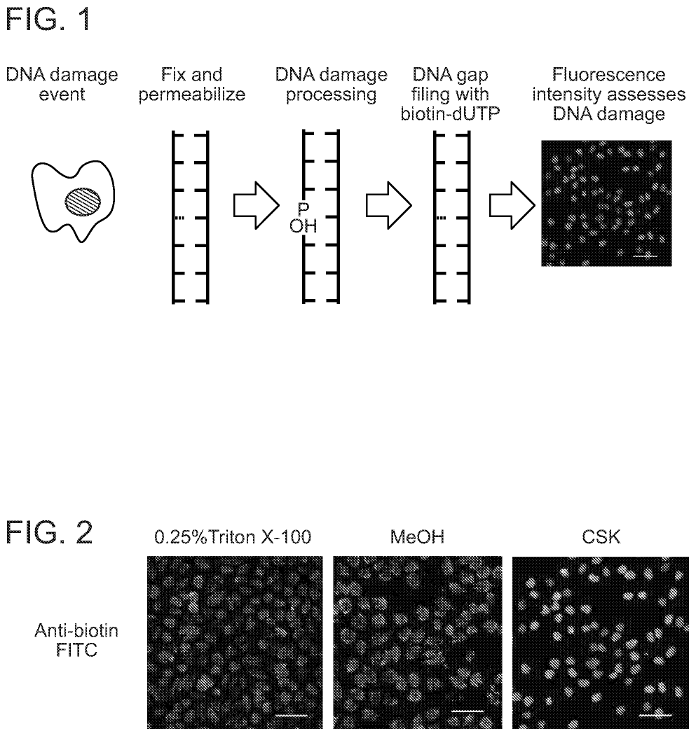

RADD is an adaptable assay capable of characterizing a broad spectrum of DNA adducts in fixed cells. Permeabilization allows the RADD enzymes access to the nucleus to process the DNA lesions present in the nuclear DNA. As depicted schematically in FIG. 1, DNA lesions are first processed by a suite of DNA repair enzymes, and the resulting gaps are then filled with a nucleotide (e.g., a biotinylated nucleotide) which allows for the DNA damage to be scored by a read-out, e.g., any fluorescent read-out, i.e., microplate readers, flow cytometry, microscopy, etc.

The RADD assay of the instant disclosure is an assay that utilizes repair enzymes capable of recognizing various oxidized bases, the modified base uracil, pyrimidine dimers, and 6-4 photoproducts (while the RADD assay has been initially exemplified using repair enzymes of bacterial origin, it is expressly contemplated that repair enzymes of non-bacterial origin can also be substituted for or added to the bacterial enzymes currently exemplified).

Without wishing to be bound by theory, the RADD assay of the instant disclosure utilizes the action of several glycosylases and endonucleases that recognize and process DNA lesions to create an appropriate substrate to be filled by DNA polymerase. The DNA end chemistry after RADD enzymatic processing is provided for both the 5′ and 3′ termini, P (phosphate), P-dimer (phosphate with covalently bound pyrimidine attached), P-UA (3′-phospho-α,β-unsaturated aldehyde), dRP (5′-deoxyribose-5-phosphate), and OH (hydroxyl). In exemplified RADD assays, the DNA polymerase I Klenow large fragment has been utilized to incorporate biotinylated dUTP at sites of DNA damage.

Endo IV of the exemplified RADD assay of the instant disclosure has two important functions in the DNA damage processing mix of the RADD assay. First, the enzyme processes apurinic/apyrimidinic (AP) sites to create the appropriate DNA end chemistry. Second, Endo IV has diesterease activity that modifies the 3′ phosphates created by the other RADD enzymes to hydroxyl groups. The large fragment of Klenow DNA polymerase, which lacks the 5′ to 3′ proofreading activity, is then used to incorporate biotinylated dUTP at the processed damage sites created by the DNA damage processing reaction (see Table 2 below). The biotinylated nucleotide provides a trackable substrate when incubated with anti-biotin FITC-conjugated antibody. The resulting biotin-dUTP repaired cells can be scored for the relative fluorescence intensity in the nucleus, which provides a measure of the DNA damage lesion load, repair efficiency and overall DDR of the cell population.

In an exemplified embodiment, the RADD assay of FIG. 1 is used to detect a broad spectrum of DNA adducts and strand breaks in cells. In this embodiment, the RADD assay is composed of two reaction cocktails. The first cocktail is a lesion processing mix (see Table 1 below), which is composed of bacterial enzymes that recognize, excise, and functionalize the DNA to allow for downstream gap-filling. These enzymes Uracil DNA glycosylase (UDG), Fapy-DNA glycosylase (FPG), T4 pyrimidine dimer glycosylase (T4 PDG), Endonuclease IV (Endo IV) and Endonuclease VIII (Endo VIII) recognize and process a large cross section of DNA adducts and free DNA ends found in cells providing users with the smallest enzyme footprint possible for detecting the totality of DNA damage in cells. Without wishing to be bound by theory, it is believed that the most important enzyme in this lesion processing mix is Endo IV, which tailors the DNA ends generated by the DNA glycosylases to ensure that a nucleotide can be efficiently inserted into the site where the DNA adduct or strand break occurred. An effective range of these enzymes was optimized based upon their activities and size to ensure they could penetrate into the nucleus of the cells of interest and process the DNA damage present. Table 1 therefore presents preferred concentrations of the lesion processing mix for this embodiment.

TABLE 1
Representative RADD Assay Sequential
Reaction Mixtures/Conditions

| Lesion | | | | | |
|---|---|---|---|---|---|
| processing | 100 μL total | | | 50 μL total | |
| mix | reaction volume | | Gap filling mix | reaction volume | |
| UDG | 2.5 | U | Large (Klenow) | 5 | U |
| | | | Fragment DNA | | |
| | | | pol I | | |
| FPG | 4 | U | Biotin-11-dUTP | 1 | μM |
| T4 PDG | 5 | U | 10 X Thermpol | 5 | μL |
| | | | buffer | | |

| | | |
|---|---|---|
| Endo IV | 5 | U |
| ENDO VIII | 5 | U |
| NAD+ | 500 | μM |
| BSA | 200 | μg/mL |
| 10 X Thermpol | 10 | μL |
| buffer | | |

While the exemplified DNA damage processing mix of the instant disclosure included 2.5 U UDG, 4 U FPG, 5U T4PDG, 5 U Endo IV, 5U ENDO VIII, 50 μM NAD⁺, 20 μg/mL BSA and 10 μL 10× Thermpol buffer, it is contemplated that, in certain embodiments, the amounts of one or more of these components can be varied without significant loss of activity. It is also the case that while the exemplified gap filling mix of the instant disclosure included 5 U Large (Klenow) Fragment DNA pol I, 1 μM Biotin-11-dUTP and 5 μL 10× Thermpol, it is contemplated that, in certain embodiments, the amounts of one or more of these components can be varied without significant loss of activity. Thus, concentration or volume within a range may be desired. Exemplary ranges of possible concentrations or volumes are presented in Table 2.

TABLE 2
Exemplary Ranges of RADD Assay Sequential
Reaction Mixtures/Conditions

| Lesion | | | | |
|---|---|---|---|---|
| processing | 100 μL total | | | 50 μL total |
| mix | reaction volume | | Gap filling mix | reaction volume |
| UDG | 1-5 | U | Large (Klenow) | 2-10 U |
| | | | Fragment DNA | |
| | | | pol I | |
| FPG | 2-10 | U | Modified-dNTP | 500 nM - 5 μM |
| T4 PDG | 2-10 | U | 10 X Thermpol | 1X working |
| | | | buffer | solution |
| Endo IV | 2-10 | U | | |
| ENDO VIII | 2-10 | U | | |
| NAD+ | 250 μm-1 mM | | | |
| BSA | 100-400 | μg/mL | | |

| | |
|---|---|
| 10 X Thermpol | 1X working |
| buffer | solution |

Further exemplary contemplated ranges for the various components of the DNA damage processing mix include the following:

| | |
|---|---|
| UDG | 1-10 U, optionally 2-4 U |
| FPG | 1-10 U, optionally 2-7 U, optionally 3-5 U |
| T4 PDG | 1-10 U, optionally 2-8 U, optionally 4-6 U |
| Endo IV | 1-10 U, optionally 2-8 U, optionally 4-6 U |
| ENDO VIII | 1-10 U, optionally 2-8 U, optionally 4-6 U |
| NAD⁺ | 50-5000 μM, optionally 200-1000 μM, |
| | optionally 400-600 μM |
| BSA | 20-2000 μg/mL, optionally 50-500 μg/mL, |
| | optionally 100-300 μg/mL |
| 10 X Thermpol | 1-100 μL, optionally 2-20 μL, optionally |
| buffer | 8-12 μL |

It is further contemplated that, apart from Endo IV, one or more of the above-recited enzymes and/or assay components could be removed from the mixture while still allowing for lesion processing to occur. Thus, in certain embodiments, at minimum, a RADD processing mixture of the instant disclosure contains Endo IV, which can detect abasic sites, followed by mixtures containing one or more enzymes selected from the group consisting of UDG, FPG, T4 PDG and ENDO VIII paired with Endo IV to detect specific classes of DNA adducts. For example, pairing T4 PDG with Endo IV can detect ultraviolet induced DNA damage, like cyclobutane pyrimidine dimers and 6,4-photoproducts, and abasic sites, and increasing the lesion mix to include T4 PDG, Endo VIII, and Endo IV can detect a wider array of damaged pyrimidine and the previous noted base lesions. Additionally, in certain embodiments, it is expressly noted that detection of spontaneous deamination events or misincorporated uracils only require UDG and Endo IV.

Further exemplary contemplated ranges for the various components of the gap filling mix include the following:

| | |
|---|---|
| Large (Klenow) | 1-10 U, optionally 2-8 U, optionally 4-6 U |
| Fragment DNA pol I | |
| Biotin-dUTP | 0.05-50 μM, optionally 0.2-5 μM, optionally |
| | 0.5-2 μM |
| 10 X Thermpol buffer | 0.5-50 μL, optionally 1-10 μL, optionally 4-6 μL |

The second mix is the gap filling mix, where a DNA polymerase can be used to insert a tagged nucleotide molecule into the gapped DNA left by the glycosylases or insert a tagged nucleotide molecule on to the ends of single and double strand breaks. In certain embodiments, the gap filling mix can be used as described in Table 1 above. However, in some embodiments, it may be desirable to utilize a concentration or volume within the range described in Table 2 above, or as otherwise recited above. It is also contemplated herein that there are a large variety of DNA polymerase molecules that can be utilized in this step, but the affinity of the polymerase of gapped DNA and DNA ends needs to be considered along with its proofreading capabilities, its ability to insert mismatched bases, and its affinity and insertion rates for modified or bulky nucleotides.

In certain exemplified embodiments, it was identified that DNA polymerase I Large (Klenow fragment), which possesses partially compromised proofreading, and the Klenow fragment exo-(with completely compromised proofreading), were the most robust enzymes for inserting nucleotides modified with small molecules (i.e., biotin or digoxigenin) or bulky fluorophore molecules (i.e., FITC, Cyanine, ALEXA, ATTO dyes) at a gaps and DNA ends; therefore, DNA polymerase I Large (Klenow fragment) is the currently recommended polymerase for the broad spectrum RADD gap filling mix. However, in other embodiments, other polymerases, may be desirable for use in the RADD assays of the instant disclosure. Select other polymerases include those of Table 3.

TABLE 3
Exemplary Polymerases
Table 3

Klenow
Bacillus stearothermophilus (Bst) (full length)
Bacillus subtilis (Bsu) Large Fragment
E. coli DNA Polymerase I
Vent (exo-) DNA polymerase

### Adduct Specific Lesion Processing Mix

In exemplified embodiments, the RADD assay has been utilized to measure a broad spectrum of DNA adducts and strand breaks, yet in another embodiment, the RADD assay can also be easily adapted to detect specific classes of DNA adducts by utilizing lesion processing mixes that exploit the substrate specificities of specific DNA glycosylases. A large number of DNA glycosylases have been identified in both bacterial and mammalian species and the DNA adducts recognized by each of these glycosylases can be utilized for more precise lesion profiling using the RADD assay, for example, if the user wants to detect specifically 8-Oxo-7,8-dihydro-2′-deoxyguanosine (8-oxodG) or 2,6-diamino-4-oxo-5-formamidopyrimidine (Fapy-G) then a lesion processing mix of Fpg only or human 8-Oxoguanine DNA Glycosylase (OGG1) can be used to detect and remove these lesions. However, OGG1 can provide users with a much more specific detection of these two lesions because Fpg also efficiently removes 4,6-diamino-5-formamidopyrimidine (Fapy-A) along with other assorted modified oxidatively-induced lesions (32-34). Further, while Fpg and OGG1 are both bifunctional DNA glycosylases that excise the damage base, the cleavage rate of abasic (AP) created by the base removal; is significant different between the two enzymes, with Fpg displaying higher activity on the AP site than OGG1 (32-34). Therefore, combining Fpg with Endo IV or OGG1 with APE1 is also contemplated to increase the overall detection rate of these lesions by increasing the removal rate of the modified base from the DNA. Therefore, depending on the adduct of interest and the known specificity of any DNA glycosylase, it is contemplated that a user can tailor a lesion processing mix for specific adduct detection using a single glycosylase (Fpg or OGG1) or combining glycosylases needed to improve the detection of different adducts and intermediate products (i.e., OGG1 and APE1). With this in mind, given any DNA adduct of interest, it is contemplated that a specific lesion processing mix can be generated by the user and utilized with the same protocol for first processing the lesions with the designed lesion processing mix, followed by gap filling to tag the processed damage site.

Other examples of tailored lesion processing mixes are: Oxidative damage detection with more specific lesions subtypes (thymine glycol or 5-hydroxycytosine) using NEIL1, NEIL2, or NEIL3; or Abasic sites detection with APE1 or Endo IV. Other specific lesion classes can be detected with more specialized glycosylases, but it is also contemplated that they would advantageously be coupled with an abasic site enzyme so there would be two lesion classes detected. However, it is contemplated that a control reaction can be run with APE1 or Endo IV alone to help identify the abasic site contributions. These include Uracil detection using UDG (UNG) with APE1 or Endo IV, UV lesions detection with T4PDG and Endo IV, and Alkylating damage detection using AAG with APE or Endo IV.

It is further contemplated that gap filling can also be tailored using different DNA polymerases as discussed above, but it is currently recommended to use a gap filling polymerase with broad specificity to ensure damage detection.

### Multiplexed Detection of DNA Adducts Using RADD Tailored Gap Filling Mixes

Although in the exemplified embodiment to measure a broad spectrum of DNA adducts and strand breaks, a modified-dUTP is utilized, it is herein contemplated that other modified nucleotides can also be used. Similar to the specific lesion mixes described above, tailored RADD gap filling mixes can also be combined with spectrally distinct or unique modified nucleotides to allow classes of DNA adducts to be specifically labeled and imaged simultaneously. In certain embodiments, the critical addition to the RADD assay that enables this multiplexed detection is the addition of a separate enzymatic reaction between the labeling events, which prevents the cross incorporation of the tagged nucleotide between lesion types.

In these embodiments of the assay, sequential complete RADD reactions (lesion processing and gap filling) are performed for the lesion class of interest using a specific tailored lesion processing mix with the damage measure using the distinct labeling strategy selected to tag each lesion class with a tailored gap filling mix (FIG. 6). For example, exposure to ultraviolet light induces both oxidative DNA damage and UV-induced bulky lesions. Therefore, it is contemplated that it may be of interest to quantify not only the broad spectrum of lesions induced but also the specific oxidative and UV components. Both oxidative and UV lesions can be mutagenic and information about their ratios may provide between information about mutagenesis rates. In this scenario, oxidatively induced DNA lesions can first be detected using a specific RADD lesion processing mix of FPG and Endo IV or OGG1 and Endo IV, then the gap filling reaction conducted with a small molecule nucleotide (i.e., biotin-dUTP) or fluorescent nucleotide (ATT0488-dUTP).

Cells can optionally then be washed. An alkaline phosphatase (shrimp, bovine or even Antarctic) enzymatic step is added to inactivate any free modified nucleotides that were not incorporated during the previous reaction (indeed, in certain (e.g., exemplified) embodiments, the phosphatase is added to avoid washing. The phosphatase can be added to the RADD mix after the first reaction in order to degrade the nucleotides, then it can be heat inactivated and the second RADD mix can be added. In certain embodiments, this step is critical because it prevents their incorporation during the second RADD reaction, which would reduce the accuracy of measuring specific DNA adducts. This step adds an additional 30 minutes to the original protocol. The cells are optionally then washed extensively to remove the alkaline phosphatase, which can be heat inactivated (indeed, phosphatase, e.g., shrimp-phosphatase can be inactivated under mild heat conditions (e.g., approximately 45° C.), and the second RADD reaction can follow without the need for an additional washing step), but if left within the cell would impede the next RADD reaction. Then a second lesion processing mix for the detection of UV-induced lesion, T4PDG and Endo IV, can be applied. In certain embodiments, Endo IV is again critical because it provides the optimal detection of the lesions by ensuring the DNA ends are compatible with the downstream gap filling reaction. Once the new RADD lesion processing mix has been applied for 1 h, then the gap filling reaction is conducted, but it is critical that a unique modified base (i.e., digoxigenin-dUTP) or spectrally distinct fluorophore (ATTO647-dUTP) is used in the second gap filling mix to precisely separate the two lesion classes. After this incorporation event, the modified nucleotides can be visualized using fluorescently labeled primary antibodies, which are spectrally separated, against biotin and digoxigenin, respectively, or the fluorescently-modified nucleotides can be immediate imaged. Relative levels of the distinct lesion types can be measured by examining the fluorescence intensity of the incorporated nucleotides.

Exemplary modified nucleotides for incorporation, e.g., into gap filling mixes of the instant disclosure include those of Table 4. US 2016/0115525, incorporated by reference herein, also recites exemplary modified and labeled nucleotides.

TABLE 4
Exemplary Modified Nucleotides

Biotin
Digoxigenin
Fluorophores: Alexa Fluor dyes, ATTO Fluor dyes, Fluorescein
dyes, Cyanine Dyes, Oxazine dyes, Rhodamine dyes
Click Chemistry: Dibenzylcyclooctyne modified, azide modified,
dibenzocyclooctyne alkyne modified

The RADD assay of the instant disclosure has been designed to allow for faster evaluation of chemical exposures and assessment of the retention of adducts in biological samples. The RADD assay acts by employing a DNA damage processing repair enzyme cocktail as described herein to detect and modify sites of DNA damage for a subsequent gap filling reaction that labels the DNA damage sites. This ability to detect and label a broad spectrum of DNA lesions within cells offers an improved (e.g., in the sense of being able to detect a broad range of DNA damage, being adaptable to high-throughput screening platforms, e.g., via direct application to fixed cells, etc.) and easy to use tool for assessing levels of DNA damage in cells that have been exposed to environmental agents or that possess natural variations in DNA repair capacity.

Because of the dynamic nature of DNA damage and repair, there is a need for creative and improved methods to characterize, quantify, and localize DNA adducts. Effective approaches have also been needed for bridging the gap between detecting single DNA adducts with extreme precision and detecting large numbers of adducts via a single method.

As demonstrated here, the RADD assay of the instant disclosure provides a dynamic, simple and accessible technique to assess a broad spectrum of DNA adducts in a cellular context over time and has been demonstrated to be capable of measuring global nuclear DNA damage (UVC, KBrO₃, and BrdU) and sub-nuclear laser micro-irradiation-induced damage with a more focused signal generation than commonly used antibody detection techniques.

Detection and quantification of DNA adducts has heretofore been technically challenging because of the large variety of DNA adducts that can be generated by endogenous and exogenous exposures. While enzyme cocktails have previously been used to detect, excise, or tailor DNA ends to improve the detection of DNA damage (T4 PDG and similar enzymes have been utilized in comet assays to improve the detection of alkali labile sites, oxidative base damage, and DNA cross-linking (11, 12) and similarly, enzyme cocktails have be used to develop modifications of the commonly used TUNEL assay to improve its detection of DNA damage (22)), such previously used examples of assays are sub-optimal, especially for in situ and/or high-throughput approaches, relative to the improvements of the RADD assays of the instant disclosure. For example, the Comet assay still requires the isolation of DNA through the application of an electric field, so the assay is not truly in situ or in vitro, in contrast to the exemplified embodiments of the instant RADD assays. Furthermore, buffer conditions of the comet assay dictate its ability to detect single strand breaks vs. double strand breaks, so the Comet assay only detects a portion of induced DNA damage. While previously described TUNEL modifications can be applied in situ or in vitro, they require a number of enzymatic steps that only increase damage detection partially because they do not include multiple glycosylases nor, more importantly, do not include Endo IV, which, without wishing to be bound by theory, is believed to be critical for the broad spectrum detection achieved by the presently exemplified RADD assays of the instant disclosure.

In view of the limited adoption of previously used assays for lesion detection and the fundamental limitations of antibody detection strategies, development of the RADD assay described herein has addressed a fundamental need for detecting and quantifying DNA adducts and DNA damage. The instant RADD assay utilizes an easy to implement enzymatic and immunofluorescence strategy that can be adopted by any laboratory and integrated into existing experimental pipelines. From fixation to imaging, the assay can be performed within 4 h, which is comparable with processing and preparation times for existing TUNEL and immunofluorescence protocols, and the RADD assay described herein is also scalable to any size desired by the user.

The optimized cell based RADD assay described herein provides improvements over previous detection methods, including enzymatic cocktails such as PreCR®, which was previously used to detect, excise, and label DNA adducts with an ATTO-conjugated dUTP upon isolated DNA (23). The instant RADD methods, at least in part, relate to discovery of modifications that improve the detection, excision, and labeling of DNA lesions within the cell (with no need for DNA isolation). In cells, assays like TUNEL have previously been found to underestimate DNA damage due to chromatin compaction, and DNA repair enzymes have been shown to have difficulty acting upon lesions that are found tightly bound by chromatin (31). To address these difficulties, stronger permeabilization and pre-extraction with CSK was employed along with sequential enzyme cocktail additions to improve the penetration and effectiveness of lesion removal in the RADD assay. By first incubating the lesion processing mix with the cells alone followed by co-incubation with the gap filling mix, the time needed for the DNA repair enzymes to diffuse into the nucleus and act on available DNA lesions is provided. Tagging of the DNA lesion site then occurs in a separate reaction after the DNA repair cocktail has provide the necessary substrate for the Klenow polymerase to fill the gap.

As will be recognized by the skilled artisan, permeabilization is distinct from fixation. Exemplified embodiments of the instantly disclosed RADD assay apply a hypotonic solution (in certain embodiments, CSK buffer for 5 min) then fix the cells with a 2% formaldehyde solution for 10 min at room temp. Permeabilization then occurs using, e.g., a 0.1% Triton X-100 in buffer for 10 min at room temp. In other embodiments, cells can be directly fixed in, e.g., a 3.7% formaldehyde solution at room temp for 10 min, then permeabilized, e.g., with 0.1% Triton X-100 for 10 min at room temp, or they can be simultaneously fixed and permeabilized, e.g., by the application of an alcohol solution for 10-15 min at −20° C. In certain embodiments, the alcohol solution has included ethanol, methanol, acetone, or 1:1 solution of methanol:acetone.

This combination of enzymatic processing and tagging was designed allow RADD to address a much wider spectrum of lesions than current adductomic or damage detection techniques and report on the overall damage state of the genome. Precise quantitation of some DNA adducts can be performed by other techniques, but the RADD assay offers facile tuning of the repair cocktail to address specific lesions (including rare adducts) given that there is an available processing enzyme. Additionally, the gap-filling reaction can also be tailored to a user's needs. In the assay described here biotinylated-dUTP was inserted and used for lesion detection however, given the ability of Klenow to insert a number of modified nucleotides, users could likely incorporate any labeled nucleotide of interest, including fluorescently-labeled nucleotides or azide/alkyne click chemistry compatible molecules. This modularity and adaptability makes RADD a powerful tool for use across many model systems, providing a single technique that is capable of characterizing many DNA repair pathways of interest.

RADD has therefore been designed and identified as an assay capable of detecting nuclear DNA lesions in cells, which can broadly detect damage across species and tissue types. The RADD assay is highly adaptable and includes: (1) a DNA damage processing mix containing DNA repair enzymes that recognize, remove, and modify DNA lesion sites to contain the appropriate DNA end chemistry for gap filling, and (2) a gap filling mix containing a tagged nucleotide to be incorporated for monitoring the processed DNA damage site. The DNA repair enzymes that are utilized by RADD can be adjusted depending upon the DNA lesions being investigated and the tagged nucleotide can also be altered depending upon the read-out that is desired. The RADD method therefore provides users with a high content screening assay capable of monitoring a broad spectrum of DNA damage with no DNA isolation required.

In certain embodiments, the disclosure encompasses kits. The kits provided may comprise a composition or agent described herein and a container (e.g., a vial, ampule, bottle, syringe, and/or dispenser package, or other suitable container). In some embodiments, provided kits may optionally further include a second container comprising an additional mixture as described herein (e.g., a second container for a gap filling mix, optionally separate from a first container for, e.g., a lesion processing mix). In some embodiments, the composition or agent described herein provided in the first container and the second container are combined to form a combined mixture for use/application/administration.

In certain embodiments, a kit described herein further includes instructions for using the kit, such as instructions for using the kit in a method of the disclosure (e.g., instructions for administering the kit to perform a RADD assay as described herein). A kit described herein may also include information as required by a regulatory agency.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Materials and Methods

### Cell Culture

A375P cells were purchased from the American Type Culture Collection (ATCC CRL-3224) and maintained in Dulbecco's modified Eagle's medium (DMEM) high glucose (Hyclone # SH30022.01) and supplemented with 10% fetal bovine serum (Atlanta Biologicals # S11550) and 1% sodium pyruvate (Gibco #11360-070). Chinese hamster ovary (CHO-K1) cells were received from Dr. Samuel H. Wilson at the National Institute of Environmental Health Sciences and grown in minimal essential medium (MEM, Hyclone # SH30265FS) supplemented with 10% FBS. All cells were maintained in a 5% CO₂ incubator at 37° C. and fewer than 10 cell passages were utilized experimentally. Mycoplasma testing was regularly performed using Lonza MycoAlert® and no contamination was detected.

### Cytotoxicity Assays

Cytotoxicity:

Cytotoxicity was determined by growth inhibition assay. A375P cells were plated at a density of 4×10⁴ cells per well in a 6-well plate (Greiner Bio-One #657165) or 35 mm dish (Falcon #353001) and treated the following day. Prior to DNA damage the cells are washed with Dulbecco's phosphate buffered saline (PBS, Cellgro #21-031-CV) or Hanks' balanced salt solution (HBSS, Hyclone # SH30031.02) for UV and KBrO₃ damage, respectively. Cells were either exposed to a specified dose of UVC (254 nm) using the Spectroline® Spectrolinker XL-1000, or exposed to potassium bromate (KBrO₃, Sigma Aldrich #309087) diluted in media for 1 h. Following the damage induction cells were washed one time in HBSS, and growth media was replaced. Cells were maintained in a 5% CO₂ incubator at 37° C. until untreated control cells reached approximately 90% confluency, typically 4-5 days. The cells were then briefly treated with 0.25% Trypsin (Life technologies #25200-056), resuspended in 1 mL of PBS, and counted with Bio-Rad TC20 automated cell counter. Results are presented as the ratio of the number of cells in treated well to cells in control well (% control survival) with error bars representing the standard error of the mean (SEM).

### Repair Assisted Damage Detection (RADD)

A375P cells were plated at 2.5×10⁶ in a 6-well plate with coverslips (VWR #48366-227) or 35 mm glass fluorodishes (World Precision Instruments # FD35-100). The following day the DNA damage was induced as described in the cytotoxicity methods with the UV and KBrO₃ doses indicated, and cells were either immediately processed for damage detection or allowed to repair for the indicated times. At the indicated time, cells were washed twice with PBS and incubated with cytoskeletal buffer (CSK, 100 mM NaCl (Fisher Scientific #7647-14-5), 300 mM sucrose (VWR #0335-1KG), 10 mM PIPES pH 6.8 (Amresco #108321-27-3), 3 mM MgCl₂ (Amresco #7786-30-3), 0.5% Triton X-100 (Sigma-Aldrich # T8787)) on ice for 5 min, followed by three washes with PBS. The samples were then incubated with 2% formaldehyde (Amresco # M134) in PBS for 10 min at room temperature (˜23° C.) and washed three times with PBS. They were next treated with 0.25% Triton X-100 (Sigma-Aldrich # T8787) in PBS for 10 min again at room temperature and washed twice with PBS, followed by one wash with sterile deionized H₂O. The DNA damage processing mix contained enzymes purchased from NEB (UDG # M0280S, Fapy-DNA glycosylase # M0240S, T4PDG # M0308S, Endo IV # M0304S, Endo VIII # M0299S) and prepared in 1× Thermpol buffer (NEB # B9004S) and incubated at 37° C. in a humidified incubator for one hour. Next, the gap filling mix, again prepared in 1× Thermpol buffer, was added to the DNA damage processing mix, and incubated for an additional hour at 37° C. Representative RADD enzymes and their functions are outlined in Table 5 (below) and representative sequential DNA damage processing and gap filling compositions and reactions are outlined in Table 1 (supra). The enzyme cocktails were then washed with 1% bovine serum albumin (BSA, Jackson ImmunoResearch #001-000-162) in PBS three times and blocked with 5% goat serum (Invitrogen #31873) in PBS for 30 minutes at room temperature. The blocking serum was then aspirated, and the goat anti-biotin FITC conjugated antibody (Sigma-Aldrich # F6762) was diluted 1:400 in 5% goat serum in PBS and incubated at room temperature for 1 h, protected from light. The cells were washed three times with 1% BSA (Jackson ImmunoReseach #001-000-162) in PBS, dried briefly, and mounted in Prolong® Gold with DAPI (Life Technologies # P36931) following the manufacturer's instructions.

### Laser Micro-Irradiation

Laser micro-irradiation was performed as previously described (24). Briefly, CHO-K1 cells were plated at 3×10⁴ cells per chamber in an 8 chamber slide (Nunc LabTek II, ThermoFisher #12-565-338) and sensitized with 10 μM bromodeoxyuridine (BrdU, Sigma Aldrich # B5002) for 24 h prior to micro-irradiation. During micro-irradiation, cells were placed in a microscope stage incubator and maintained at 37° C. with 5% CO₂. A 405 nm laser (Coherent Obis) coupled to a Nikon Alrsi laser scanning confocal microscope was used to induce DNA damage by creating a damage region of interest (ROI) consisting of a 3×3 pixel area that was stimulated at 0.5 frames per second (fps) through a 20× C-Apochromat (NA 0.75) dry objective. The post objective laser output was measured using a PM100D power meter equipped with a S170C objective plane power sensor (THORLabs # S170C and S150C) and 0.5 fps stimulation resulted in 2.4 mW.

### Immunofluorescence

Following micro-irradiation, cells were fixed and permeabilized in 100% ice cold methanol for 15 min followed by 5 washes with PBS to allow cells to fully rehydrate. Prior to probing for the DNA strand break proteins cells were blocked in 1% BSA in PBS for 30 min at room temperature. Cells were then incubated with the primary antibodies for γH2AX (Millipore 05-363) and XRCC1 (abcam ab1838) diluted in 1% BSA in PBS at a 1:750 and a 1:50 ratio, respectively, for 1 h at room temperature. For the cyclobutane pyrimidine dimer (CPD) immunofluorescence, cells were fixed and rehydrated as described, and then the DNA was denatured using 2N hydrochloric acid (HCl, Fisher # SA49) for 45 min at room temperature and washed 5 times with PBS. Samples were then neutralized in 50 mM Tris-HCl pH 8.8 (Amresco # J383) for 5 min at room temperature and washed 3 times with PBS. Samples were blocked in 5% normal goat serum (Pierce #31873) for 30 min at room temperature followed by an incubation with anti-CPD (Cosmo Bio clone TDM2) in 5% goat serum for 1 h at room temperature. All samples were then washed 3 times with PBS and incubated with Alexa-488 goat anti-mouse (ThermoFisher # A11034) in 1% BSA for 1 h at room temperature protected from light. Samples were washed 3 times with PBS and the nucleus was stained using NucBlue® fixed cell stain (DAPI, ThermoFisher # R37606) following the manufacturer's instructions.

### Imaging and Image Analysis

Fluorescence images were acquired using the Nikon Alrsi laser scanning confocal microscope using a 20× dry objective (numerical aperture 0.75). Multi-channel configuration was used to ensure the absence of excitation cross-talk or emission bleed-through between channels. The gain of the 405 nm laser line was set so the nucleus was clearly defined following DAPI staining. To image the DNA damage sites labeled with the FITC-anti-biotin antibody (RADD signal), the 488 nm laser gain was set to 2.5 for all quantitative imaging acquisition. The gain setting was determined by first examining cells processed with both the DNA damage and gap filling mixes, though the Klenow was omitted from the gap-filling step. Imaging of the no Klenow processed cells revealed a low level of non-specific staining in the cytoplasm and no nuclear staining (FIG. 8), and the gain was set to eliminate this non-specific signal. This method was previously employed to address non-specific antibody staining for signal quantitation (25). Then the untreated, but completely processed control cells were examined and gain was adjusted to image their relative intensity to just above the background. 2-D images were acquired, and DAPI staining was used to select the largest cross section of the nucleus for imaging. Images were acquired with a pinhole of 3 airy units (AU) and a zoom of 1.0. NIS-Elements AR 4.51 software was used for all image acquisition.

Image analysis was performed using the Fiji ImageJ software package (see fiji.sc). Images were cropped to eliminate partial cells on the borders and cells not in the focal plane. Images were not cropped more than 30% of the original picture size 512×512. The RADD signal was scored using the DAPI channel to define the nucleus and applying that mask to the FITC channel in order to measure the mean nuclear signal intensity of the anti-biotin-FITC conjugated antibody in the nucleus. A minimum threshold was applied to select nuclei, and analyze particles was used to filter nuclei sizes between 50-1000 μm to prevent large aggregates from being analyzed. The representative images and graphs were constructed from multiple image fields from separate biological replicates (n≥3), with the error bars representing SEM.

### Example 2: RADD Assay Development

The RADD assay was exemplified as an assay that utilized repair enzymes of bacterial origin capable of recognizing various oxidized bases, the modified base uracil, pyrimidine dimers, and 6-4 photoproducts, as depicted in Table 5.

TABLE 5
Representative RADD Assay Enzymes

| | | Processed terminal ends | |
|---|---|---|---|
| Enzyme | Function | 5′ | 3′ |
| Uracil DNA glycosylase | Monofunctional glycosylase that catalyzes the removal of | AP site | |
| | uracil from DNA | | |
| Formamidopyrimidine [Fapy]- | bifuntional glycosylase that recognizes and removes various | P | P |
| DNA glycosylase | types of oxidized purines such as 8-oxoguanine | | |
| T4 Pyrimidine dimer glycosylase | Bifunctional glycosylase that recognizes and removes | P-imer | P-UA |
| | cyclobutane pyrimidine dimers and 6-4 photoproducts | | |
| Endonuclease IV | Apurinic/apyrimidinic endonuclease that modifies AP lesion | dRP | OH |
| | and diesterease activity modifies 3′ phosphates | | |
| Endonuclease VIII | Bifunctional glycosylase that can catalyze the removal of | p | P |
| | various damaged pyrimidines | | |

| | | |
|---|---|---|
| Klenow DNA polymerase large | Incorporates biotin dUTP at sites created by DNA adduct | DNA synthesis 5′ → 3′ |
| fragment | processing mix including overhangs | |

Uracil DNA glycosylase (UDG), Fapy-DNA glycosylase (FPG), T4 pyrimidine dimer glycosylase (T4 PDG), and Endonuclease VIII (Endo VIII) recognize and process a large cross section of DNA adducts found in cells; however, the processed DNA ends are not always compatible for DNA synthesis. It was identified that for the RADD reaction disclosed herein to successfully label these sites of DNA damage, further processing by Endonuclease IV (Endo IV) was needed to create the appropriate 3′ hydroxyl group required for the subsequent gap filling reaction.

The RADD assay was performed in two sequential reactions without aspirating reagents between reactions. RADD assay reaction components were combined in the following mixtures:

The exemplified DNA damage processing mix (which included 2.5 U UDG, 4 U FPG, 5U T4 PDG, 5 U Endo IV, 5 U ENDO VIII, 500 μM NAD⁺, 200 μg/mL BSA and 10 μL 10× Thermpol buffer—see Table 1 above) was placed on fixed and permeabilized cells and placed in a humidified incubator. The exemplified gap filling mix (which included 5 U Large (Klenow) Fragment DNA pol I, 1 μM Biotin-11-dUTP and 5 μL 10× Thermpol—see Table 1 above) was added directly to the lesion processing mix and incubated for an additional hour. The reagents were then aspirated and the cells were washed and incubated with anti-biotin FITC conjugated antibody.

### Example 3: Disrupting the Cytoplasm Increased Nuclear RADD Signal

The enzymatic load of the DNA damage processing and gap filling reaction cocktails of the exemplified RADD assay were designed to reach the nucleus and act upon exposed DNA lesions, thereby allowing for efficient detection of DNA lesions in cells. Several permeabilization techniques were investigated in a number of cell lines (including A375P, U2OS, HCT116, CHO-K1) to optimize the nuclear fluorescence signal of RADD. Among cell lines examined, the melanoma cell line, A375P, was identified as the most difficult to permeabilize and detect damage in, so it was used as a model cell line for RADD assay optimization work. As shown in FIG. 2, standard permeabilization techniques with both ice cold methanol and 0.25% Triton X-100 allowed for some detection of DNA lesions within the nucleus, but the signal was low in the nuclear compartment, and many cells accumulated a halo surrounding the nuclear envelope. However, as also shown in FIG. 2, cells that were treated with CSK, a hypotonic solution, followed by mild permeabilization prior to RADD contained a much more uniform and robust nuclear fluorescence signal. Thus, treatment of cells with CSK buffer removed much of the cytoplasm and likely allowed the RADD enzymes a less encumbered route when diffusing into the nucleus.

### Example 4: RADD Detected Lesions Induced by Oxidizing Agents and UV

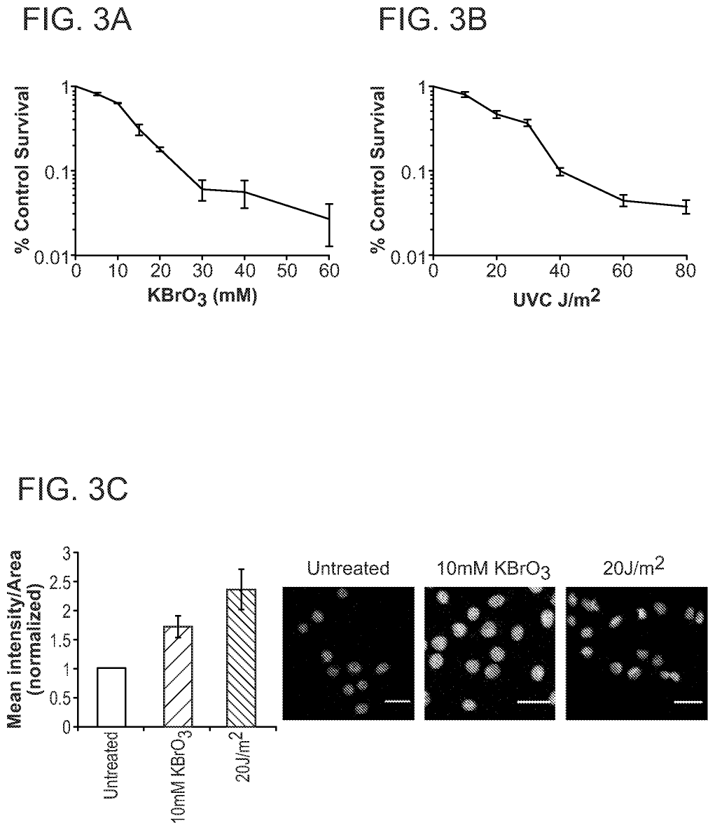

To validate detection of induced DNA damage and assess the sensitivity of the RADD assay exemplified herein, cells were expose to two common environmental damaging agents prior to RADD analysis. DNA damage was induced in A375P cells by exposing them to the oxidizing agent KBrO₃ or to UVC light. Sensitivity of the A375P cell line to these agents was first characterized by cell growth inhibition. As shown in FIG. 3A, KBrO₃ induced cell death in the mM range, with near complete cell growth inhibition at concentrations greater than 30 mM. As shown in FIG. 3B, UVC exposure fully inhibited cell growth at doses above 60 J/m².

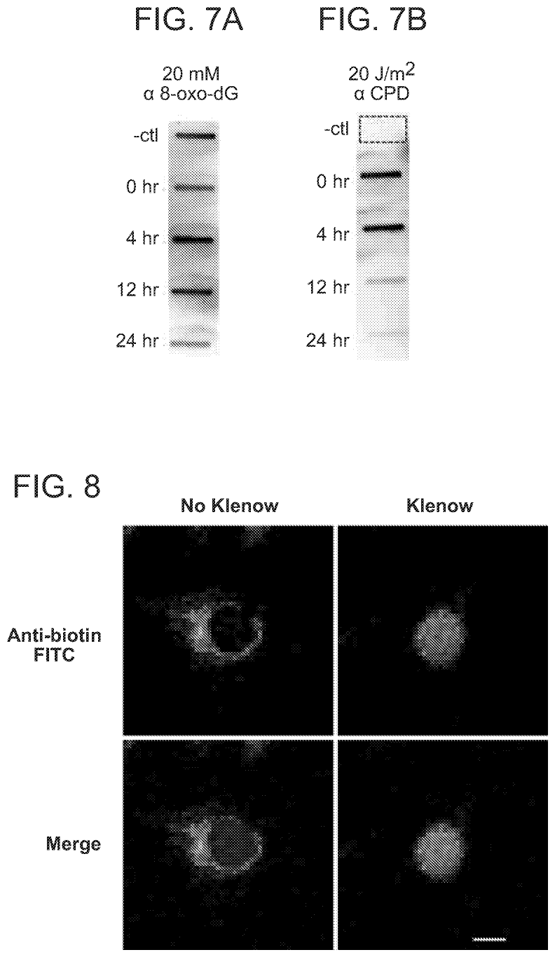

Based on the cell sensitivity curves, the doses of 10 mM KBrO₃ (64% growth inhibition) and 20 J/m² UVC (48% growth inhibition) were used to validate the ability of RADD to detect DNA damage created by these treatments. Verification of the induction of DNA adducts by KBrO₃ and UV treatment was confirmed by slot blot analysis of the genomic DNA (FIGS. 7A and 7B). RADD was able to detect the induced DNA damage created by both treatments with relative fluorescence signal intensities of 1.7× and 2.3× above untreated cells for KBrO₃ and UV induced damage, respectively. These results demonstrated that RADD was capable of detecting not only the basal levels of DNA damage in the cells, but also the induction of nuclear DNA damage in the forms of oxidized bases, such as 8-oxo-2′-deoxyguanosine (8-oxodG), created by 10 mM KBrO₃ treatment for 1 h, as well as UV induced bulky adducts, such as CPD.

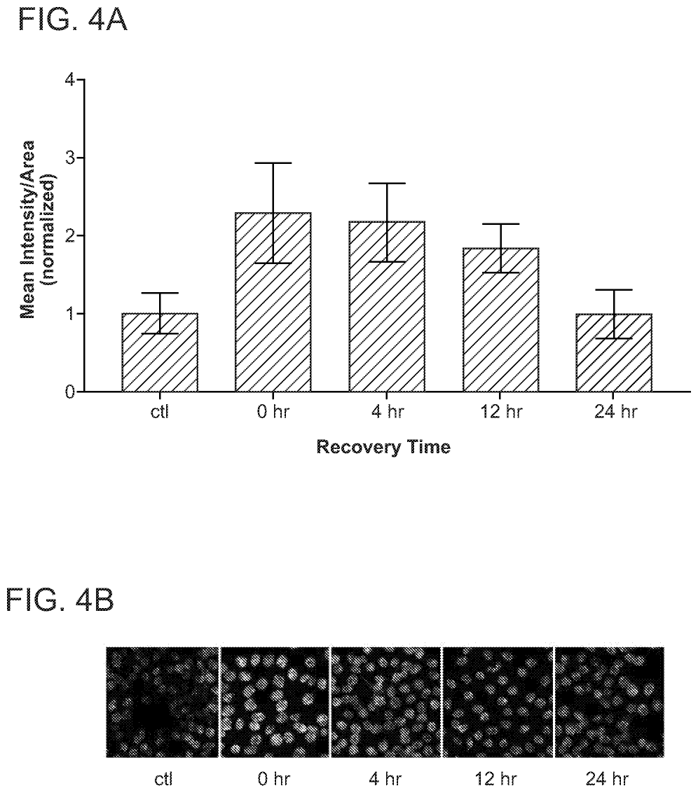

As shown in FIGS. 4A and 4B, repair of these lesions was also monitored by performing a RADD time course after damage induction and monitoring the changes in fluorescent signal as a function of time (FIG. 4). A375P cells were treated with 10 mM KBrO₃ for 1 h, then RADD was performed after allowing cells to repair for 4, 12, and 24 h. RADD was able to detect not only the induction of lesions (0 time point), but was also able to show their removal as a function of repair time, demonstrated by the reduction in fluorescence signal over the time course. The selected dose of 10 mM KBrO₃ was mildly cytotoxic to the A375P cells, so return to basal levels of DNA damage after 24 h was consistent with cells surviving the induced DNA damage.

### Example 5: RADD Detected the Base Lesion BrdU and DNA Damage Induced by Laser Micro-Irradiation

Laser micro-irradiation has emerged as a powerful and commonly used tool to characterize DDR following a sub-nuclear dose of laser irradiation. DDR has been investigated by performing micro-irradiation at wavelengths ranging from UV to near infra-red, with most wavelengths inducing mixtures of base damage and single and double strand DNA breaks (26, 27). The use of sensitizing agents like BrdU has been employed to reduce the dose of laser irradiation needed to elicit a DDR. BrdU is a modified nucleotide that is an analog of thymidine and is inserted into the DNA of actively replicating cells. Without wishing to be bound by theory, the increase in sensitization is the result of the BrdU being spontaneously liberated from the DNA backbone by UVB or UVA excitation, producing an abasic site (28). Additionally, specific laser wavelengths have also been shown to produce reactive oxygen species when interacting with BrdU moieties to produce oxidative damage in addition to the base loss. To examine if the RADD assay could detect the incorporation of BrdU and detect the DNA damage site created by laser micro-irradiation, CHO-K1 cells were employed (the laser micro-irradiation damage response of CHO-K1 cells was previously examined (24)).

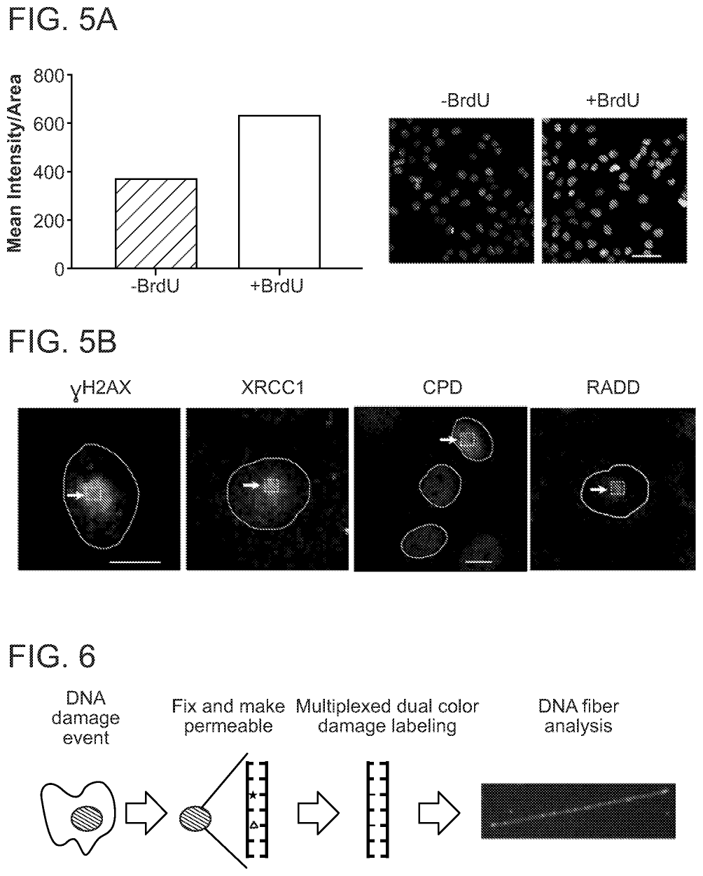

CHO-K1 cells were first sensitized for 24 h with BrdU, and incorporation of BrdU into the genomic DNA was probed using RADD. As shown in FIG. 5A, basal levels of DNA damage in the CHO-K1 cells were detected using RADD, and cells exposed to BrdU showed increased levels of DNA lesions, as detected by the increase in mean fluorescence intensity of the nucleus.

Laser micro-irradiation was then used to induce DNA damage in these cells. Laser-induced DNA damage has been characterized by observing the recruitment and retention of DNA repair proteins to the site of induced damage, via fluorescently-tagging or immunofluorescence, and the types of DNA damage induced have often been characterized by immunofluorescent detection of strand break markers, such as XRCC 1, γH2AX or 53BP-1, or adducts, such as 8-oxodG, CPD, or 6-4 photoproducts. Here, the RADD assay was used to detect the induction of DNA damage in the CHO-K1 cells 5 min post micro-irradiation, and the RADD detected spot was compared to the commonly used strand break and damage markers.

As shown in FIG. 5B, CHO-K1 cells irradiated at 0.5 fps showed large γH2AX foci that covered much of the nucleus, showing signal propagation from the site of induced damage. As shown in FIG. 5B, the single strand break and base excision repair protein XRCC1 also showed recruitment to the induced damage site, and while distinct foci did form, the signal was still diffuse. When the RADD assay was performed upon cells that were micro-irradiated, the localization of the fluorescent signal was tightly correlated with the damage ROI used to create the laser induced DNA damage. Further, as also shown in FIG. 5B, the RADD-detected signal was also significantly better than the lesion detection antibody for CPD, which required denaturing of the DNA to generate the diffuse spot observed.

Thus, the RADD assay demonstrated the ability to characterize the damage created by both the modified DNA base used to sensitize the reaction and the laser micro-irradiation itself. It therefore provided a distinct advantage over adduct antibodies, which may perform poorly and require DNA to be denatured for recognition, and strand break signals, like γH2AX, which has been shown to develop in the absence of detectable DSBs or develop pan-nuclear signaling making break sites difficult to detect (29, 30).

### CITATIONS OF PUBLICATIONS REFERENCED HEREIN

1. Negrini, S., V. G. Gorgoulis, and T. D. Halazonetis, Genomic instability—an evolving hallmark of cancer. Nat Rev Mol Cell Biol, 2010. 11(3): p. 220-8.
2. Olaussen, K. A., et al., DNA repair by ercc 1 in non-small-cell lung cancer and cisplatin-based adjuvant chemotherapy. N Engl J Med, 2006. 355(10): p. 983-91.
3. de Boer, J. and J. H. Hoeijmakers, Nucleotide excision repair and human syndromes. Carcinogenesis, 2000. 21(3): p. 453-60.
4. Akbari, M. and H. E. Krokan, Cytotoxicity and mutagenicity of endogenous DNA base lesions as potential cause of human aging. Mech Ageing Dev, 2008. 129(7-8): p. 353-65.
5. Jalal, S., J. N. Earley, and J. J. Turchi, DNA repair: From genome maintenance to biomarker and therapeutic target. Clin Cancer Res, 2011. 17(22): p. 6973-84.PMC3218201.
6. Balbo, S., R. J. Turesky, and P. W. Villalta, DNA adductomics. Chem Res Toxicol, 2014. 27(3): p. 356-66.PMC3997222.
7. Figueroa-Gonzalez, G. and C. Perez-Plasencia, Strategies for the evaluation of DNA damage and repair mechanisms in cancer. Oncol Lett, 2017. 13(6): p. 3982-3988.PMC5452911.
8. Guo, J., et al., Multiclass carcinogenic DNA adduct quantification in formalin-fixed paraffin-embedded tissues by ultraperformance liquid chromatography-tandem mass spectrometry. Anal Chem, 2016. 88(9): p. 4780-7.PMC4854775.
9. Dizdaroglu, M., P. Jaruga, and H. Rodriguez, Measurement of 8-hydroxy-2′-deoxyguanosine in DNA by high-performance liquid chromatography-mass spectrometry: Comparison with measurement by gas chromatography-mass spectrometry. Nucleic acids research, 2001. 29(3): p. E12.30413.
10. Valavanidis, A., T. Vlachogianni, and C. Fiotakis, 8-hydroxy-2′-deoxyguanosine (8-ohdg): A critical biomarker of oxidative stress and carcinogenesis. J Environ Sci Health C Environ Carcinog Ecotoxicol Rev, 2009. 27(2): p. 120-39.
11. Olive, P. L. and J. P. Banith, The comet assay: A method to measure DNA damage in individual cells. Nat Protoc, 2006. 1(1): p. 23-9.
12. Smith, C. C., M. R. O'Donovan, and E. A. Martin, Hoggl recognizes oxidative damage using the comet assay with greater specificity than fpg or endoiii. Mutagenesis, 2006. 21(3): p. 185-90.
13. Braafladt, S., V. Reipa, and D. H. Atha, The comet assay: Automated imaging methods for improved analysis and reproducibility. Sci Rep, 2016. 6: p. 32162.PMC5007470.
14. Collins, A. R., et al., Controlling variation in the comet assay. Front Genet, 2014. 5: p. 359.PMC4202776.
15. Collins, A. R., et al., The comet assay: Topical issues. Mutagenesis, 2008. 23(3): p. 143-51.
16. Didenko, V. V., In situ labeling of DNA breaks and apoptosis by t7 DNA polymerase. Methods Mol Biol, 2011. 682: p. 37-48.PMC3324588.
17. Hornsby, P. J. and V. V. Didenko, In situ ligation: A decade and a half of experience. Methods Mol Biol, 2011. 682: p. 49-63.PMC3324560.
18. Loo, D. T., Tunel assay. An overview of techniques. Methods Mol Biol, 2002. 203: p. 21-30.
19. Didenko, V. V., H. Ngo, and D. S. Baskin, Early necrotic DNA degradation: Presence of blunt-ended DNA breaks, 3′ and 5′ overhangs in apoptosis, but only 5′ overhangs in early necrosis. Am J Pathol, 2003. 162(5): p. 1571-8.PMC1851179.
20. van Dierendonck, J. H., DNA damage detection using DNA polymerase i or its klenow fragment. Applicability, specificity, limitations. Methods Mol Biol, 2002. 203: p. 81-108.
21. Otsuki, Y. and Y. Ito, Quantitative differentiation of both free 3′ oh and 5′ oh DNA ends using terminal transferase-based labeling combined with transmission electron microscopy. Methods Mol Biol, 2002. 203: p. 41-54.
22. Baskin, D. S., M. A. Widmayer, and M. A. Sharpe, Quantification ofdnase type i ends, dnase type ii ends, and modified bases using fluorescently labeled ddutp, terminal deoxynucleotidyl transferase, and formamidopyrimidine-DNA glycosylase. Biotechniques, 2010. 49(1): p. 505-12.
23. Zirkin, S., et al., Lighting up individual DNA damage sites by in vitro repair synthesis. J Am Chem Soc, 2014. 136(21): p. 7771-6.
24. Holton, N. W., J. F. Andrews, and N. R. Gassman, Application of laser micro-irradiation for examination of single and double strand break repair in mammalian cells. p. e56265.
25. Kirby, T. W., et al., DNA polymerase beta contains a functional nuclear localization signal at its n-terminus. Nucleic Acids Res, 2017. 45(4): p. 1958-1970.PMC5389473.
26. Lukas, C., et al., Distinct spatiotemporal dynamics of mammalian checkpoint regulators induced by DNA damage. Nat Cell Biol, 2003. 5(3): p. 255-60.
27. Gassman, N. R. and S. H. Wilson, Micro-irradiation tools to visualize base excision repair and single-strand break repair. DNA Repair (Amst), 2015. 31: p. 52-63.PMC4458156.
28. Limoli, C. L. and J. F. Ward, A new method for introducing double-strand breaks into cellular DNA. Radiat Res, 1993. 134(2): p. 160-9.
29. Cleaver, J. E., L. Feeney, and I. Revet, Phosphorylated h2ax is not an unambiguous marker for DNA double-strand breaks. Cell Cycle, 2011. 10(19): p. 3223-4.
30. Rybak, P., et al., Low level phosphorylation of histone h2ax on serine 139 (gammah2ax) is not associated with DNA double-strand breaks. Oncotarget, 2016. 7(31): p. 49574-49587.PMC5226530.
31. Campalans, A., et al., Interaction with oggl is required for efficient recruitment of xrccl to base excision repair and maintenance of genetic stability after exposure to oxidative stress. Mol Cell Biol, 2015. 35(9): p. 1648-58.PMC4387214.
32. Sidorenko, V. S. and D. O. Zharkov, Correlated cleavage of damaged DNA by bacterial and human 8-oxoguanine-DNA glycosylases. Biochemistry, 2008. 47(34): p. 8970-6.
33. Zharkov, D. O., et al., Substrate specificity and reaction mechanism of murine 8-oxoguanine-DNA glycosylase. J Biol Chem, 2000. 275(37): p. 28607-17.
34. Watson, A. J. and G. P. Margison, Assays for the repair of oxidative damage by formamidopyrimidine glycosylase (fpg) and 8-oxoguanine DNA glycosylase (ogg-1). Methods Mol Biol, 2000. 152: p. 17-32.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The present disclosure teaches one skilled in the art to test various combinations and/or substitutions of chemical modifications described herein toward generating conjugates possessing improved contrast, diagnostic and/or imaging activity. Therefore, the specific embodiments described herein are not limiting and one skilled in the art can readily appreciate that specific combinations of the modifications described herein can be tested without undue experimentation toward identifying conjugates possessing improved contrast, diagnostic and/or imaging activity.

The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

What is claimed is:

1. A composition for detecting and processing DNA adducts contained in damaged DNA, comprising a plurality of two or more enzymes selected from the group consisting of uracil DNA glycosylase (UDG), formamidopyrimidine [Fapy]-DNA glycosylase (FPG), T4 pyrimidine dimer glycosylase (T4PDG), endonuclease IV (Endo IV), and endonuclease VIII (ENDOVIII), provided that one of the plurality of enzymes is Endo IV.

2. The composition of claim 1, which comprises at least three of said enzymes.

3. The composition of claim 1, which comprises at least four of said enzymes.

4. The composition of claim 1, which comprises all five of said enzymes.

5. A kit for conducting repair assisted Damage detection (RADD), comprising the composition of claim 1, and instructions for its use.

6. The RADD kit of claim 5, wherein each enzyme is contained in a separate container.

7. The RADD kit of claim 5, wherein each enzyme is contained in the same container.

8. The RADD kit of claim 5, further comprising a gap filling mix disposed in a further separate container, wherein the gap fill mix comprises a Klenow DNA polymerase and deoxy Uracil Triphosphate (dUTP) which may be directly or indirectly conjugated to a label.

9. A method of detecting and quantifying damage to nuclear DNA, comprising:
a) fixing and permeabilizing cells;
b) treating the fixed and permeabilized cells with a composition for detecting and processing DNA adducts which comprises a plurality of two or more enzymes selected from the group consisting of UDG, FPG, T4PDG, ENDOIV, and ENDOVIII, provided that one of the plurality of enzymes is ENDOIV, wherein the treating recognizes and processes DNA adducts contained in the nuclear DNA, thus allowing incorporation of a labelled dUTP into the adduct;
c) treating the cells of b) with a gap filling mix comprising a Klenow DNA polymerase and dUTP, wherein the dUTP is labeled, or a label is affixed thereto following incorporation of the dUTP into the damaged nuclear DNA; and
d) detecting and quantifying the DNA damage as a function of intensity of the label.

10. The method of claim 9, wherein the cells are eukaryotic in origin, optionally wherein the cells are selected from the group consisting of human cancer cells A375P, U2OS, HCT116 and mammalian CHO-K1 cells.

11. The method of claim 9, wherein prior to the permeabilizing, the cells are incubated in a hypotonic solution.

12. The method of claim 11, wherein the hypotonic solution comprises a cytoskeleton buffer.

13. The method of claim 11, wherein after application of the hypotonic buffer, cells are fixed with a formaldehyde solution and further permeabilized with mild permeabilization buffer, optionally wherein the mild permeabilization buffer is a 0.25% Triton X-100 buffer solution.

14. The method of claim 9, wherein b) comprises incubating the fixed and permeabilized cells with UDG, FPG, T4PDG, ENDOIV, and ENDOVIII.

15. The method of claim 9, wherein the dUTP is linked to biotin, and wherein the detecting and quantifying in d) comprises incubating the cells of c) with an anti-biotin antibody, and then quantifying the DNA damage as a function of the intensity of the label that is indirectly conjugated to the anti-biotin antibody.

16. The method of claim 15, wherein the label is a fluorescent dye, and the quantifying comprises measuring the intensity of the fluorescent signal emitted by the dye, optionally wherein measuring the intensity of the fluorescent signal emitted by the dye comprises detection of a fluorescent read-out, optionally wherein the fluorescent read-out is selected from the group consisting of microscopy, plate reader(s) and flow cytometry.
